(19) United States
(12) Patent Application Publication
(10) Pub. No.: US 2020/0030527 A1
(43) Pub. Date: Jan. 30, 2020

(54) FLUID MANAGEMENT SYSTEM

(71) Applicant: Hermes Innovations LLC, Cupertino, CA (US)

(72) Inventors: Akos Toth, Cupertino, CA (US); Csaba Truckai, Saratoga, CA (US)

(73) Assignee: Hermes Innovations LLC, Cupertino, CA (US)

(21) Appl. No.: 16/435,323

(22) Filed: Jun. 7, 2019

Related U.S. Application Data
(60) Provisional application No. 62/684,672, filed on Jun. 13, 2018.

(51) Int. Cl. A61M 5/145; A61M 5/168

(52) U.S. Cl. CPC A61M 5/14593; A61M 2039/0009; A61M 5/16854

(57) ABSTRACT

A surgical fluid management system includes a console and a cassette for delivering fluids to a surgical site. The console has a pump rotor and a pressure-sensing membrane. The cassette has a cassette housing, a flexible fluid delivery tube in the housing. The flexible fluid delivery tube has a lumen configured to interface with the pump rotor and to deliver a flow of fluid from a fluid source as the rotor is rotated. A pressure-transmitting membrane is located in a wall of the cassette housing and in fluid communication with said fluid delivery lumen,. The pressure-transmitting membrane flexes outwardly in response to a positive pressure in the lumen and flexes inwardly in response to a negative pressure in the lumen. The pressure-transmitting membrane detachably adheres to or presses against the pressure-sensing membrane to cause the pressure-sensing membrane to move in response to pressure changes in the flexible fluid delivery tube.

### CROSS-REFERENCE

This application claims the benefit of Provisional Application No. 62/684,672 (Attorney Docket No. 37644-716.101), filed on Jun. 13, 2018, the full disclosure of which is incorporated herein by reference.

### BACKGROUND OF THE INVENTION

### 1. Field of the Invention

The present invention relates generally to a surgical fluid management system and more particularly to a surgical fluid management system of the type used in endoscopic procedures.

Surgical fluid management systems typically deliver a fluid, such as saline, to a targeted working space or body cavity to provide access and visibility to the physician performing a procedure in the working space or body cavity. The fluid usually provides a pressure sufficient to “open” the space (i.e. create a working space for the procedure) as well as flushing blood and debris from the space. Typically, the surgical fluid management system includes a control system for maintaining a preset fluid pressure in a working space.

Surgical fluid management systems are often inconvenient to use and difficult to monitor. Further, the control systems of such fluid management systems are often unable to accurately measure pressure in a working space when the patient and the fluid management console are at different elevations.

It would therefore be beneficial to provide improved surgical fluid management systems that overcome at least some of these shortcomings. In particular, it would be desirable to provide surgical fluid management systems with an improved ability to measure pressure in a patient working space and to utilize the improved pressure measurements for determining changes in elevation of a surgical tool delivering a surgical fluid to the working space. At least some of these objectives will be met by the inventions described below.

### 2. Listing of Background Art

US20160242844; US20180326144; and US20190030235 have common inventorship and describing surgical fluid management systems.

### SUMMARY OF THE INVENTION

In general, the fluid management system includes a disposable cassette carrying inflow and/or outflow tubing sections that are configured for releasably mating with a control unit and roller pump head(s). The fluid management system can be adapted to automatically recognize the type of disposable cassette and the volume of fluid in an inflow source. During operation, the system can calculate pressure in the working space based on fluid pressure in the cassette tubing set, and provide for inflow and outflow control to maintain a desired pressure in the working space or adjust other operating parameters. Other features, objects, and advantages will be apparent from the description and drawings, and from the claims.

The present invention provides improved fluid management systems and methods for their use. In particular, the present invention provides a disposable tubing cassette, a consoles for detachably receiving the disposable tubing cassette, and methods for mounting and replacing the tubing cassette on the console. The disposable tubing cassette will usually include a first flexible tubing loop, where the tube is used for delivering fluid from a fluid source to a patient. A second tube may be used for removing fluid from the patient and delivering the fluid to a disposal receptacle. The fluid management systems may also be configured to alert the user when the cassette has been successfully loaded or, conversely, when the cassette has not been successfully loaded. Further capabilities include sensing conditions of the fluid, in particular, positive and negative pressures in a fluid in an inflow pathway of the cassette. Automatic locking capabilities may also be provided by a motor and control mechanism carried by the console.

In a first specific aspect, the present invention provides a disposable cassette for use with a surgical fluid management system having a console with a peristaltic pump rotor. The cassette comprises a housing, a flexible tube located in the housing configured to engage the peristaltic pump rotor when the cassette is mounted on the console.

In other specific embodiments, the disposable cassette may further comprise a flexible membrane on a sensing window on at least one of the first and second flexible tubes. The at least one sensing window will usually be positioned to align with a pressure or force sensor on the console when the cassette is mounted on the console. In an exemplary embodiment, the membrane of the sensing window comprises a thin resilient element overlying an interior chamber in a housing that communicates with a fluid flow path in inflow tubing carried by the cassette. In a specific embodiment, the pressure sensor in the console is mounted on a sliding base plate that carries the cassette.

In another specific embodiment, the cassette membrane and cooperating flexible membrane of the pressure sensor in the console are adapted to maintain contact with one another to thus allow measuring both positive and negative pressures in a fluid column in the cassette. The ability to measure negative pressures in the cassette is relevant when the treatment tool in the console are different elevations which then allows for more precise calculation of the actual fluid pressure in the working space. In one specific embodiment, the cassette membrane and the sensor membrane carry magnets or magnetic response material to allow for detachable coupling of the services of the membranes.

In one particular aspect of the present invention, a cassette for use in a surgical fluid management system comprises a cassette housing, a flexible tube in the housing, and a pressure-transmitting membrane in a wall of the cassette housing. The cassette is intended for use in a surgical fluid management system which typically includes a console with a pump rotor and a pressure-sensing membrane. The flexible tubing in the cassette housing has a lumen configured to interface with the pump rotor of the console and to carry a fluid from a fluid source, typically to a surgical tool being used in a patient working space. The pressure-transmitting membrane in the cassette housing is in fluid communication with the lumen of the flexible tubing, and typically the pressure-transmitting membrane is configured to flex outwardly in response to a positive pressure in the flexible tube lumen and to flex inwardly in response to a negative pressure in the flexible tube lumen. To help assure that the pressure of the surgical fluid in the fluid tubing is accurately transmitted to the console of the surgical fluid management system, the pressure transmitting membrane will be configured to detachably adhere to or to press against and deform the pressure-sensing membrane when the cassette is received on the pump rotor.

Configuring the pressure-transmitting membrane to detachably adhere to and/or to press against and deform the pressure-sensing membrane is advantageous in that such enhanced proximity will improve the accuracy of pressure transmission across the adjacent membranes and can be achieved in a number of specific ways. For example, the pressure-transmitting membrane may comprise a magnetic material configured to magnetically couple to a magnetic material in the pressure-sensing membrane. The phrase “magnetic material” includes both permanently magnetic materials, e.g. permanent magnets, and magnetizable materials, i.e. those which are magnetized and attracted to a permanent magnet. At least one of the magnetic materials in the pressure-transmitting membrane and the pressure-sensing membrane will usually be a permanent magnet, while the other of the membranes may possess either a permanent magnet or a magnetizable material.

Alternatively, the pressure-transmitting and/or the pressure-sensing membrane may be modified to include an adhesive coating on the surface that interfaces with the surface of the adjacent membrane. Suitable adhesive coatings include synthetic setai of the type which adhere to an adjacent surface via van der Waals forces. Low tack adhesives, such as the type used on sticky notes, may also be used. Either or both membranes may be coated with an adhesive lubricant, typically an oil-based lubricant of the type which has an inherent adhesive quality.

As a further alternative to magnetic materials and adherent materials, the pressure-sensing and/o pressure-transmitting membrane may comprise a suction adhesion element, such as a suction cup, configured to couple to the adjacent membrane surface.

As a still further alternative to coatings and mechanical attachment elements, the pressure-transmitting membrane in the cassette and/or the pressure-sensing membrane in the console may be deformed to bow or otherwise extend outwardly from a flat configuration. In this way, the deformed membrane will engage and deform the adjacent membrane such that an elastic recoil of the adjacent membrane will act to more closely conform to the adjacent membrane to enhance coupling and pressure/force transmission.

In preferred instances, the cassette housing will have a chamber therein which is in fluid communication with the lumen of the flexible tubing. The chamber will act as a reservoir for the fluid being delivered through the flexible tubing, and the pressure-transmitting membrane may comprise a wall of the chamber.

In an additional particular aspect of the present invention, a surgical fluid management system comprises a cassette as generally described above, in combination with a console having a pump rotor and a pressure-sensing membrane. The console may comprise a force-sensing element or pressure sensor, and one or more elements may be provided which project inwardly from a back surface of the pressure-sensing membrane to engage the force-sensing element. Such elements both transmit the force from the pressure-sensing membrane to the force or pressure-sensing element, and further act to deform a front surface of the pressure-sensing membrane outwardly. The outwardly extending (bowed) front surface of the pressure-sensing membrane may act to deform the pressure-transmitting membrane on the cassette inwardly to enhance contact between the membranes.

In a further particular aspect, the present invention provides a surgical fluid management system including a console and a cassette housing. A flexible tube is disposed in the cassette housing and has a lumen configured to interface with the pump rotor to carry a flow fluid from a fluid source. A pressure-transmitting membrane is formed in a wall of the cassette housing and is in fluid communication with the lumen of the flexible tubing. The pressure-transmitting membrane is configured to flex outwardly in response to a positive pressure in the lumen and to flex inwardly in response to a negative pressure in the lumen. A force sensing element in the console has one or more elements which project inwardly from a back surface of the pressure-sensing membrane to engage a force-sensing element, where one or more elements which project inwardly from a back surface of the pressure-sensing element deform a front surface of the pressure-sensing membrane outwardly to enhance contact between the adjacent surfaces of the two membranes.

In another particular aspect of the present invention, a surgical fluid management console is provided for use with a cassette having flexible tubing configured to interface with a pump rotor and a pressure-transmitting membrane. The surgical fluid management console includes a pump rotor configured to receive the flexible tubing of the cassette, a pressure-sensing membrane, and a force-sensing element. The pressure-sensing membrane is configured to engage the pressure-transmitting membrane when the flexible tubing is mounted on the pump rotor. One or more elements project inwardly from a back surface of the pressure-sensing membrane to engage the force-sensing element, where the one or more elements deform a front surface of the pressure-sensing membrane outwardly to engage and deform the pressure-transmitting membrane inwardly to enhance contact without said membranes.

In yet another particular aspect, the present invention provides a method for managing fluids during a medical procedure. The method comprises providing a fluid management console having a pump rotor, a pressure-sensing membrane, and a pressure sensor coupled to the pressure-sensing membrane. A cassette is also provided, where the cassette has a pressure-transmitting membrane and a flexible tube configured to receive fluid from a fluid source and to interface with the pump rotor. The cassette is removably mounted on the fluid management console in such a way that the pump rotor rotatably engages the flexible tubing, and the pressure-sensing membrane on the console engages the pressure-transmitting membrane on the cassette. Sufficient contact force between the two membranes is provided to enhance the transmission of pressure from the flexible tubing through the two membranes, to the pressure sensor in the console. The pump rotor may then be rotated to pressurize and deliver fluid from a fluid source through the flexible tubing, and the pressure sensor will be able to accurately generate a signal representative of a pressure in the flexible tubing.

In specific instances, the pressure sensor will be able to detect and measure both positive and negative pressure in the flexible tubing of the cassette, where positive and negative are conveniently measured relative to an initial pressure often set at the outset of a procedure. In more specific instances, the pressure-transmitting membrane and the pressure-sensing membrane may be adapted to flex outwardly and inwardly, when engaged against each other, in response to positive pressure and negative pressure, respectively, in the flexible tubing. In further specific instances, the console may be adapted to calculate a change in elevation of a treatment device delivering a fluid from the flexible tubing to a patient working space receiving fluid from the flexible tubing. Such calculations will typically be based upon a positive and/or negative pressure signal from the pressure sensor in the console. Conveniently, the positive and negative pressure signals may be based on a value zeroed at the beginning of the procedure when the membranes are in a neutral, un-stressed configuration.

### BRIEF DESCRIPTION OF THE DRAWINGS

FIG. 1 is a perspective view of a console or control unit of a fluid management system in phantom view that includes an inflow peristaltic pump and a detachable cassette that carries an inflow tubing loop adapted for engaging the peristaltic pump head.

FIG. 2 illustrates a back side of the cassette of FIG. 1 further showing a flexible membrane of a sensing window in the fluid inflow path of the cassette adapted to interface with a pressure sensor membrane of the console of FIG. 1 further showing the fixed base plate that carries the pump motors and a portion of the sliding base plate.

FIG. 3A is an enlarged schematic view of the flexible cassette membrane interfacing with a sensor membrane of a pressure sensor carried by the console in a first static condition, wherein both membranes carry magnets for detachable coupling of the membranes.

FIG. 3B is a schematic view of the membranes of FIG. 3A showing the flexible cassette membrane flexing outwardly relative to the cassette in response to positive fluid pressure in the fluid inflow path which flexes the sensor membrane and allows the sensor elements to calculate the positive pressure.

FIG. 3C is a view of the membranes of FIG. 3B showing the flexible cassette membrane flexing inwardly relative to the cassette in response to negative fluid pressure in the fluid inflow path which flexes the sensor membrane and allows the sensor elements to calculate the negative pressure.

FIG. 4 is an enlarged view of another variation of a flexible cassette membrane that interfaces with a sensor membrane, wherein the cassette membrane carries at least one suction cup element for detachable coupling of the cassette and sensor membranes.

FIG. 5A is an enlarged schematic view of another variation of flexible cassette membrane that interfaces with a sensor membrane that has a projecting feature that contacts a pressure or force sensor in a first static condition.

FIG. 5B is a view of the sensor membrane of FIG. 5A showing the flexible cassette membrane flexing outwardly relative to the cassette in response to positive fluid pressure in the fluid inflow which flexes the sensor membrane and the projecting feature or element into the force sensor allowing calculation of the positive pressure.

FIG. 5C is a view of the sensor membrane of FIG. 5A showing the flexible cassette membrane flexing inwardly relative to the cassette in response to negative fluid pressure which flexes the sensor membrane and the projecting feature or element away from the force sensor allowing calculation of the negative pressure.

### DETAILED DESCRIPTION OF THE INVENTION

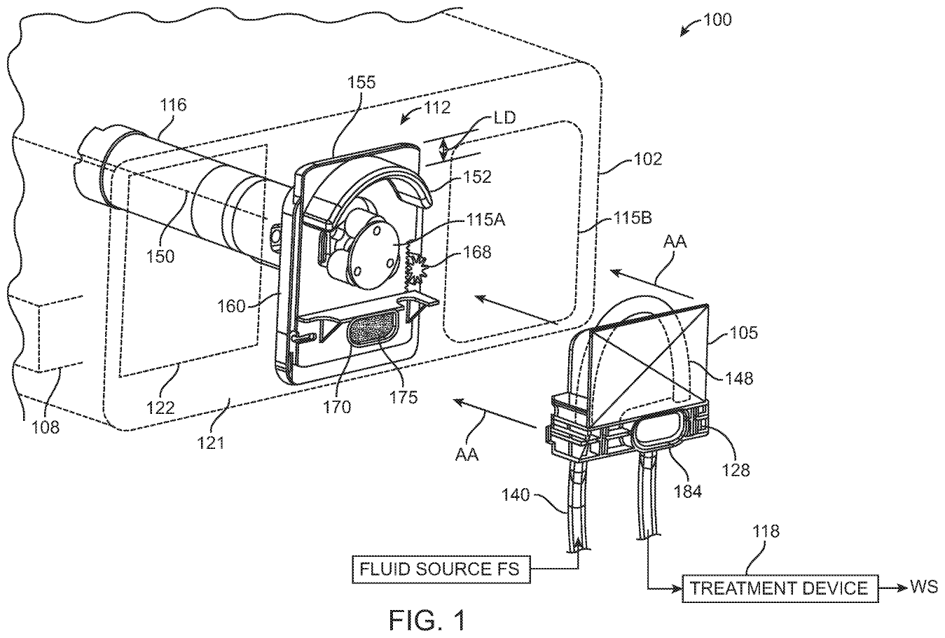

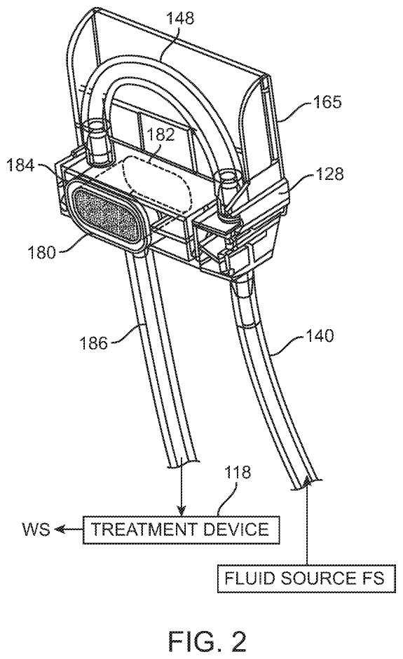

FIG. 1 illustrates a fluid management system 100 of the invention which includes a console or control unit 102 and a disposable tubing cassette 105 (FIGS. 1-2) that carries a single loop of an inflow tubing set for coupling to an inflow pump 115A further described below. The fluid management system 100 is used in endoscopic procedures, which can be a urology surgery, gynecology procedure or arthroscopic surgery, to provide inflows and outflows of a pressurized fluid to a working space or body cavity. The fluid can be delivered to provide and maintain a pre-set pressure level within the working space. The fluid pressure in the space is controlled by a controller 108 and control algorithms therein carried by the control unit 102 which can calculate the fluid pressure in the working space based on sensed pressure in a fluid inflow path at the control unit 102 and then vary the inflow and/or outflow to maintain a targeted pressure or a targeted pressure in combination with an inflow or outflow rate. An outflow pump mechanism is not shown in detail, which could be a second pump 115B (phantom view) in the control unit 102 or wall suction could be used.

Referring to FIGS. 1-2, the console or control unit 102 carries a first peristaltic pumps 112 comprising pump head 115A with rollers and a motor 116 (see FIG. 1) wherein the pump provides inflows from a fluid source FS into a working space WS. Typically, the fluid inflows and outflows are provided through one or more channels in an endoscope and/or a treatment device 118. The control unit 102 includes a microprocessor or controller 108 for controlling the inflow pump and may further include an RF generator or other energy source for coupling to a treatment device 118 and/or a power source to powering a motor in the treatment device 118.

In FIG. 1, one variation of control unit 102 has a front surface 121 which can include a touch screen (not shown) that permits the operator to control system operations. For example, the touch screen 122 can allow the operator select a target pressure, flow rate and/or mode of operation. In one variation described further below, the touch screen 122 can indicate when the user positions the cassette 105 in the correct interface with the control unit 102, and thereafter the control unit can automatically activate a locking motor to engage and move the cassette 105 from a pre-locked position to a locked position to engage the pump heads as will be described below. In these steps, the touch screen 122 can display the pre-locked and locked positions. The touch screen 122 can then be touched to actuate the locking motor to unlock the cassette 105 following a procedure. In other variations, the cassette 105 can be manually inserted and pushed into a locked positioned. It has been found that significant manual force may be required to push the cassette 105 into a locked position, and the amount of force may vary depending on the orientation of the rollers in the pump heads 115A and 115B, and for this reason a motorized locking system may be preferred.

Referring to FIGS. 1-2, the cassette 105 includes a plastic molded housing or body 128 that carries portions of a tubing set, and more particularly a flexible loop of inflow tubing 140. The tubing is typically a flexible polymer material having a diameter ranging between about ¼″ to ½″ and is adapted to cooperate with the first and second pump heads 115A and 115B (see FIG. 1). The tubing loop portion 148 in the cassette 105 (see FIGS. 1-2) extends in a semicircular arc of at least 90° or at least 120° in the plane of the cassette, where the plane of the cassette is adapted to align with the first pump head 115. As shown in FIG. 2, the plane of the tubing loop portions is perpendicular to the axis 150 of a shaft of the pump motor 116 and the pump head 115A.

Referring to FIG. 1, it can be seen as the tubing loop portion 148 within the cassette 105 is adapted to be inserted between the pump head 115A (roller assemblies) and the arcuate structure or eyebrow 152 that interfaces with the tubing loop 148 and pump head 115A.

From FIG. 1, it can be understood how the cassette 105 is coupled with the control unit 102. The cassette 105 is initially pushed inward toward the front panel 121 of the control unit 102 as indicated by arrows AA. The tubing loop portion 148 of the inflow tubing is then loosely positioned in the space between eyebrow 152 and the pump head 115A.

It can be understood that after inserting the cassette 105 and tubing loop over the pump head 115A, it is necessary to compress the tubing loop portion 148 between the pump head 115A and the eyebrow 152 which is be accomplished by the downwards sliding movement of the sliding base plate 155 which carries eyebrows 152 and the cassette 105. The pump head 115 and motor 116 are attached to the fixed base plate 160 which is coupled to the front panel 121 of the control unit 102 (FIG. 1). As can be understood from FIG. 1, the sliding base plate 155 and eyebrow 152 together with the cassette 105 can be moved downward a locking distance indicated at LD which thus compresses the tubing loop portion 148 between the eyebrows 152 and the pump head 115A.

A locking motor (not visible) with a gear reduction mechanism rotates a gear 168 that is adapted to move the sliding base plate 155 the locking distance LD to thereby move the cassette 105 from a pre-locked position to a locked position. The locking motor can be activated by microswitch (not shown) in the console 102 or sliding base plate 155 that is activated when the cassette 105 is pushed inwardly against the sliding base plate 155.

Still referring to FIG. 1, it can be seen that the sliding base plate 155 carries a pressure sensor 170 with a sensor membrane 175 that is adapted to contact a flexible membrane 180 carried by the cassette 105 (see FIG. 2). In FIG. 2, it can be seen that the cassette membrane 180 is disposed on a side of a fluid chamber 182 in the cassette that communicates with fluid inflows or static fluid in the inflow tubing 140. As can be understood from FIG. 2, the flow path in the inflow tubing 140 extends through a housing 184 that carries the fluid chamber 182 and the cassette membrane 180 is adapted to flex inwardly and outwardly depending on pressure of the fluid in the chamber 182 and the lumen 186 of the inflow tubing 140. Thus, the flexible membrane 180 carried by the cassette 105 interfaces with the pressure sensor membrane 175 carried by the sliding base plate 155. Some similar pressure sensing mechanisms are known in the prior art. However, in this variation, the interface of the cassette membrane 180 and pressure sensor membrane 175 differ in that the membranes 175 and 180 are aligned in direct opposition to one another after the cassette 105 is pushed onto the pump head 115A and thereafter the membranes 175 and 180 remain in a non-sliding or fixed relationship as the sliding base plate 155 is moved to compress the tubing loop 148 against the pump head 115A.

By measuring fluid pressure with such a sensor mechanism in the control unit 102, the fluid pressure in the working space can be calculated, which is known in the prior art. Of particular interest in the present invention, the pressure sensing mechanism corresponding to the invention is configured to allow the pressure sensor 170 carried by the sliding base plate 155 to sense positive pressure in the fluid inflows as well as negative pressure. Prior art systems were designed only for sensing positive pressure in a fluid inflow.

In some surgical procedures such as gynecology, it is important to regulate or maintain “actual” fluid pressure in a working space WS within a narrow predetermined range or a not-to-exceed pressure. Further, it can be understood that the elevation of pump head 115A relative to the patient and the working space WS can make the fluid pressure in a working space different from the measured pressure in the cassette 105. In other words, the “actual” fluid pressure in a working space WS will differ from the pressure sensed at the control unit 102 simply based on the elevation difference between the control unit 102 and the working space WS. For example, in a gynecology procedure, the variance in the height of the control unit 102 relative to the working space WS can result in a sensed pressure at the control unit 102 that varies by up to 10% or more from the actual pressure in the working space WS. Over the time of a surgical procedure, such an inaccurate pressure measurement can be problematic and potentially cause injury to the patient by such overpressure in the working space WS.

Thus, in a typical procedure after the patient is prepared for surgery and the working space WS is filled with fluid and the tubing sets have been purged of air, a difference in elevation of the treatment device 118 or working space WS relative to the console 102 can be calculated by a positive or negative pressure reading the pressure sensor 170 which interfaces with the cassette membrane 180.

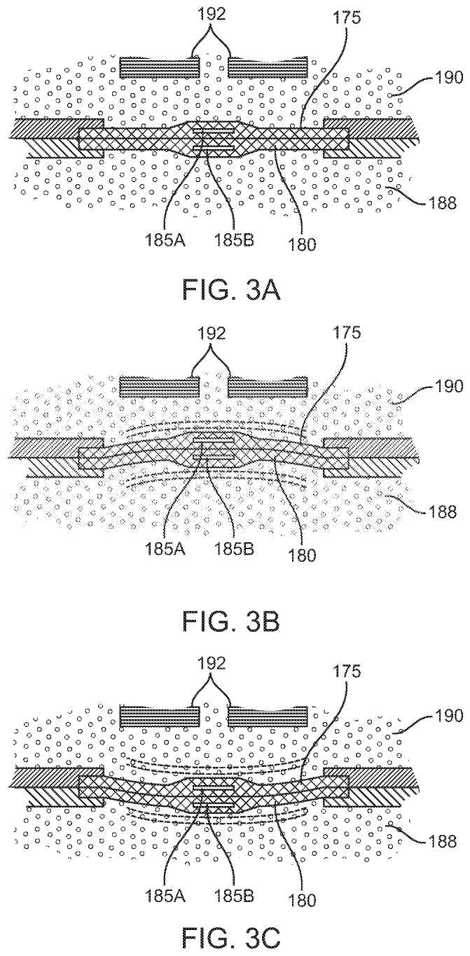

In order for the sensor membrane 180 to measure negative pressures, or flex inwardly relative to the cassette, a mechanism is provided to detachably adhere the cassette membrane 180 to the sensor membrane 175. Now referring to FIG. 3A, in one variation, the sensor membrane 175 and the cassette membrane 180 each carried a magnet 185a, 185b (or a magnetic response material in one membrane that is attracted to a magnet in the other membrane). Thus, in FIG. 3B, it can be seen that a positive pressure in the fluid 188 against the cassette membrane 180 flexed the sensor membrane 175 and the increased pressure in fluid 190 in the sensor is read by the sensing elements 192. In FIG. 3C, it can be seen that if is negative pressure in the fluid 188 in the cassette inflow path, then the cassette membrane 180 will flex inwardly relative to the cassette wherein such a negative pressure influences the sensor membrane 175 which again can sensed by the sensing elements 192. Prior to the procedure, the sensing elements 192 can be zeroed-out to have a baseline value, and thereafter the elevation of the treatment device 118 and the working space WS relative to the console 102 can be determined by positive pressure as illustrated in FIG. 3B or by negative pressure as illustrated in FIG. 3C.

FIG. 3A-3C show a first magnet 185a in the sensor membrane 175 and a second magnet 185b in the cassette membrane 180, but it should be appreciated that a single magnet in one membrane and magnetic responsive material such as iron powder can be dispersed in the second membrane to insure that the membranes 175, 180 remain coupled to one another whether there is positive or negative pressure in the fluid 188 in the inflow path in the cassette 105.

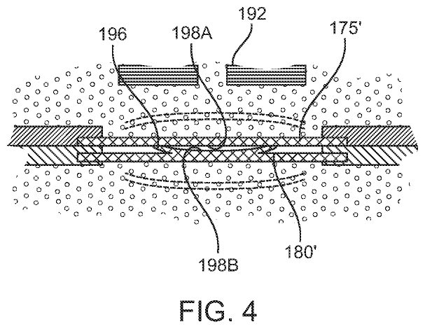

FIG. 4 illustrates another variation which couples the sensor membrane 175′ with the cassette membrane 180′ which comprises at least one flexible suction cup element 196 that detachably couples together the exterior surfaces one 198a and 198b of the two membranes 175′ and 180′. It should be appreciated that other mechanisms are possible for detachably coupling the membranes, such as providing one membrane surface with microfabricated synthetic setae of the type developed to mimic setae on gecko's feet. As is well known, gecko setae are adapted to detachably contact and adhere to smooth surfaces. In another variation, the surface of the cassette membrane 180 may be covered with removable protective element, and the membrane surface can be provided with a slightly tacky adhesive similar to a Post-It in order to allow for detachably coupling of the two membranes 175, 180. In another variation, the cassette membrane may be covered by a removable protective element and the membrane surface can carry a viscous fluid or grease that is sufficient to maintain adherence between the two membranes during use.

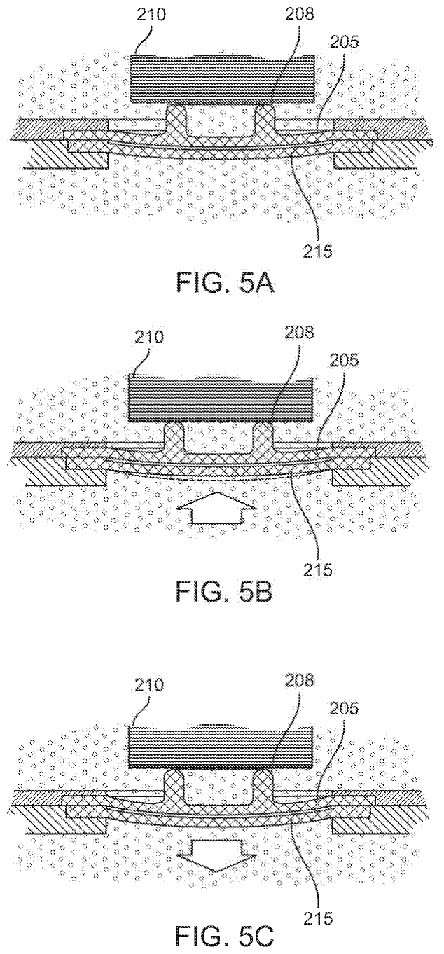

Now turning to FIG. 5A-5C, another variation of sensing mechanism is shown wherein the sensor membrane 205 includes a projecting feature or element 208 that contacts a force sensor element 210 and wherein in a repose position, the sensor membrane 205 is flexed outwardly. Thereafter, following the locking of the cassette 105 and the sliding base plate 155 as described previously, the cassette membrane 215 will be flexed inwardly (relative to the cassette 105) in response to the outward bulging of the sensor membrane 205. Thus in FIG. 5B, it can be seen that positive pressures in the flow path and cassette can cause both membranes 205, 215 to flex in the direction of the force sensing element 210 and wherein the force sensor can determine the positive pressure. Referring to FIG. 5C, the opposite is also possible where a negative pressure in the fluid in the inflow path results in the membrane sensor membrane 205 flexing outwardly relative to the console 102 which then can be read by the force sensing element 210 to calculate the negative pressure.

The console 102 carries a controller 108 with a microprocesser that operates in accordance with algorithms to control inflows and outflows of a fluid to a working space to maintain a pre-set pressure level within the space. The console 102 can further include an RF generator or other energy source for coupling to a surgical instrument. The system optionally can monitor pressure in a space directly with a pressure sensor in a fluid communication with the space through an open channel in a device which then will allow the controller 108 to vary inflows and/or outflows to maintain the targeted pressure.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A cassette for use in a surgical fluid management system having a console with a pump rotor and a pressure-sensing membrane, said cassette comprising:
a cassette housing;
flexible tubing in the housing having a lumen configured to interface with the pump rotor and to carry a flow of fluid from a fluid source; and
a pressure-transmitting membrane in a wall of the cassette housing and in fluid communication with said lumen, said pressure-transmitting membrane being configured to flex outwardly in response to a positive pressure in the lumen and to flex inwardly in response to a negative pressure in the lumen;
wherein the pressure-transmitting membrane is further configured to detachably adhere to or press against and deform the pressure-sensing membrane when the cassette is received on the pump rotor.

2. The cassette of claim 1, wherein the pressure-transmitting membrane comprises a magnetic material configured to magnetically couple to a magnetic material in the pressure-sensing membrane.

3. The cassette of claim 2, wherein the magnetic material comprises a permanent magnetic material.

4. The cassette of claim 2, wherein the magnetic material comprises a magnetizable material.

5. The cassette of claim 1, wherein pressure-transmitting membrane comprises an adhesive coating on a surface that interfaces with a surface of the pressure-sensing membrane.

6. The cassette of claim 5, wherein the adhesive coating comprises synthetic setae which adhere to the adhesive coating via van der Waals forces.

7. The cassette of claim 5, wherein the adhesive coating comprises a low tack adhesive.

8. The cassette of claim 1, wherein pressure-sensing membrane comprises a suction adhesion element.

9. The cassette of claim 1, wherein pressure-sensing membrane comprises an adhesive lubricant.

10. The cassette of claim 1, wherein the pressure-transmitting membrane is deformed outwardly to press against and deform the pressure-sensing membrane inwardly.

11. The cassette of claim 1, further comprising a chamber in the housing in fluid communication with the lumen, wherein the pressure-transmitting membrane comprises a wall of the chamber.

12. A surgical fluid management system comprising:
a console having a pump rotor and a pressure-sensing membrane; and
a cassette as in claim 1.

13. The surgical fluid management system of claim 12, further comprising a force sensing element in the console and one or more elements which project inwardly from a back surface of the pressure-sensing membrane to engage the force sensing element.

14. The surgical fluid management system of claim 13, wherein the one or more elements deform a front surface of the pressure-sensing membrane outwardly to engage and deform the pressure-transmitting membrane inwardly to enhance contact between said membranes.

15. A surgical fluid management system comprising:
a console having a pump rotor and a pressure-sensing membrane;
a cassette housing;
flexible tubing in the cassette housing having a lumen configured to interface with the pump rotor and to carry a flow of fluid from a fluid source;
a pressure-transmitting membrane in a wall of the cassette housing and in fluid communication with said lumen, said pressure-transmitting membrane being configured to flex outwardly in response to a positive pressure in the lumen and to flex inwardly in response to a negative pressure in the lumen; and
a force sensing element in the console has one or more elements which project inwardly from a back surface of the pressure-sensing membrane to engage a force sensing element, wherein the one or more elements which project inwardly from a back surface of the pressure-sensing membrane deform a front surface of the pressure-sensing membrane outwardly to engage and deform the pressure-transmitting membrane inwardly to enhance contact between said membranes.

16. A surgical fluid management console for use with a cassette having flexible tubing configured to interface with a pump rotor and a pressure-transmitting membrane, said surgical fluid management console comprising:
a pump rotor configured to receive the flexible tubing of the cassette;
a pressure-sensing membrane configured to engage the pressure-transmitting membrane when the flexible tubing is mounted on the pump rotor; and
a force sensing element;
one or more elements which project inwardly from a back surface of the pressure-sensing membrane to engage the force sensing element, wherein the one or more elements deform a front surface of the pressure-sensing membrane outwardly to engage and deform the pressure-transmitting membrane inwardly to enhance contact between said membranes.

17. A method for managing fluids during a medical procedure, said method comprising:
providing a fluid management console having a pump rotor, a pressure-sensing membrane, and a pressure sensor coupled to the pressure-sensing membrane;
providing a cassette having a pressure-transmitting membrane and a flexible tubing configured to receive fluid from a fluid source and interface with the pump rotor;
mounting the cassette on the fluid management console so that the pump rotor rotatably engages the flexible tubing and the pressure-sensing membrane on the console engages the pressure-transmitting membrane on the cassette with sufficient contact to transmit pressure in the flexible tubing from the pressure-transmitting membrane to the pressure-sensing membrane; and
rotating the pump rotor to pressurize and deliver fluid from a fluid source through the flexible tubing;
wherein the pressure sensor generates a signal representative of a pressure in the flexible tubing.

18. A method as in claim 17, wherein the pressure sensor measures positive and negative pressure in the flexible tubing of the cassette.

19. The method of fluid management of claim 18, wherein the pressure-transmitting membrane and the pressure-sensing membrane are adapted to flex outwardly and inwardly in response to positive pressure and negative pressure, respectively, in the flexible tubing.

20. The method of fluid management of claim 17, further comprising calculating a change in elevation of a treatment device delivering a fluid from the flexible tubing to a working space receiving a fluid from the flexible tubing based upon a positive or negative pressure signal from the pressure sensor.

21. The method of fluid management of claim 20, wherein the pressure signal from the pressure sensor is zeroed at the beginning of a procedure.
